(19) United States
(12) Patent Application Publication
(10) Pub. No.: US 2018/0172700 A1
(43) Pub. Date: Jun. 21, 2018

(54) Affinity Plate for Haptoglobin Phenotype Determination, Kit Comprising It, and Method of Haptoglobin Phenotype Determination by Means of Affinity Plates in Combination with Desorption Ionization Mass Spectrometry Techniques

(71) Applicants: Petr Novak, Dolni Brezany (CZ); Michael Volny, Praha 4 (CZ); Petr Pompach, Mratin (CZ); Viktor Ruzicka, Rymarov (CZ)

(72) Inventors: Petr Novak, Dolni Brezany (CZ); Michael Volny, Praha 4 (CZ); Petr Pompach, Mratin (CZ); Viktor Ruzicka, Rymarov (CZ)

(21) Appl. No.: 15/736,855

(22) PCT Filed: Jun. 16, 2016
(86) PCT No.: PCT/CZ2016/000067

§ 371 Date: Dec. 15, 2017

(30) Foreign Application Priority Data
Jun. 16, 2015 (CZ) PV 2015-405

(51) Int. Cl. G01N 33/68; C12P 21/06; C07K 14/47

(52) U.S. Cl. CPC G01N 33/6851; G01N 33/6893; C12P 21/06; H01J 49/0418; G01N 2333/4713; G01N 2560/00; C07K 14/4717

(57) ABSTRACT

The invention relates to the affinity plate for haptoglobin phenotype determination, the kit comprising it, and the method of haptoglobin phenotype determination by detection of its a subunits by means of mass spectrometry desorption ionization techniques after preceding preconcentration of haptoglobin on the surface of the affinity plate with immobilized anti-haptoglobin antibody that was deposited onto this surface from gas phase after preceding electrospray ionization and heat desolvation. Then the signals of mass spectra corresponding to α1 and/or α2 subunits can be found and thus determined the haptoglobin phenotype, whereas only al subunit exists in phenotype 1-1, both the subunits a1 and α2 exist in phenotype 2-1, and only α2 subunit exists in phenotype

### FIELD OF THE INVENTION

The invention relates to the affinity plate for haptoglobin phenotype determination, the kit comprising it, and the method of haptoglobin phenotype determination with the use of desorption ionization mass spectrometry techniques.

### BACKGROUND OF THE INVENTION

Haptoglobin (Hp) is one of the most abundant glycoproteins secreted by liver into the blood plasma. Its concentration varies in the range of 0.4 g/L to 2 g/L at healthy adult. Haptoglobin belongs to the proteins called acute phase proteins. Its most important biological role is to capture free haemoglobin that causes oxidative stress and damages of surrounding tissues. Haptoglobin binds the haemoglobin molecule via very strong bond (K_{d}≈1×10⁻¹⁵ mol/L) and the resulting complex is then specifically recognized by macrophages, where it is degraded and the liberated hem group is transferred to bilirubin.

Haptoglobin monomer molecule consists of two subunits: the larger one β subunit and smaller one a subunit that are bound to each other via disulphide bridges. Human haptoglobin molecule is encoded by one gene localized on chromosome 16. Two alleles formed by partial duplication of the gene of a chain give three haptoglobin variants: Hp(1-1), Hp(2-1), and Hp(2-2). Phenotype 1-1 consists of a dimer formed by one α1 and one β subunit, phenotype 2-1 consists of α1β dimer and α2β dimer, and phenotype 2-2 consists of α2 and β subunit. Phenotypes 2-1 and 2-2 form noncovalent multimers of high molecular weight varying in the range of 300 kDa to 900 kDa (Boonyapranai, K. et al. Glycoproteomic analysis and molecular modeling of haptoglobin multimers. Electrophoresis 32, 1422-32 (2011). Andersen, C. B. F. et al. Structure of the haptoglobin-haemoglobin complex. Nature 489, 456-9 (2012)).

The distribution of the phenotype of particular forms of haptoglobin differs in the worldwide population. The highest frequency of α1 allele was found in South African and South American population, on the contrary, the lowest frequency was observed in Asian population. In Europe, the distribution of haptoglobin phenotypes in the population is approximately as follows: Hp1-1—13%, Hp2-1—49%, and Hp2-2—38%. Similar distribution of haptoglobin phenotypes was observed in North American and Canadian population. On the contrary, in Asian population, the phenotype Hp1-1 is represented by approximately 9%, phenotype Hp2-1 by 37%, and phenotype Hp2-2 by 54%, depending on demographical distribution ((Langlois, M. & Delanghe, Clin. Chem. 1600, 1589-1600 (1996)).

In the past, haptoglobin phenotype diagnostic was used in forensic medicine for disproving paternity. Nowadays it has been revealed that haptoglobin phenotype plays an essential role in a number of diseases, thus it can be used as a predictive biomarker. At cardiovascular diseases, the patients with phenotype 2-2 were indicated to have shorter survival time after surgical bypassing of the narrowed or closed part of the artery than the patients with phenotype 1-1. Phenotype 2-2 is also associated with higher incidence of atherosclerotic plaque at patients with ischemic heart disease. Diabetic patients with phenotype 1-1 show up to 5 times better survival prognosis at cardiovascular diseases than the diabetic patients with phenotype 2-2. Similarly the patients with phenotype 1-1 are better protected against restenosis after coronary artery stent implantation (Sadrzadeh, S. M. H. & Bozorgmehr, Pathol. Patterns Rev. 121, 97-104 (2004)).

Several methods are used for haptoglobin phenotype determination, based on genotyping, separation, and affinity chromatography. Zone electrophoresis was one of the oldest methods used for the identification of haptoglobin phenotype. In the course of time, several other methods, using different variations of gel electrophoresis, were developed. Amersham Biosciences Company commercially presented a method of haptoglobin phenotype determination with the use of PhastSystem based on the separation of the haptoglobin-haemoglobin complex by means of native gradient polyacrylamide gel followed by the Malachite green staining detection in combination with hydrogen peroxide (Hansson, L. et al. Application Note 373. 1-4).

Thanks to the peroxidase activity of haptoglobin-haemoglobin complex, one band of dimer can be observed on the gel for phenotype 1-1, trimer and tetramer for phenotype 2-1. The formation of higher multimers is observed for haptoglobin with phenotype 2-2, whereas dimer is not formed. This method is time consuming and experimentally difficult, both during the sample preparation (it is necessary to mix the serum and the hemolysate in the suitable appropriate ratio to form the complex), and in the realization itself. The use of toxic dyestuffs Malachite green and Rhodamine presents another substantial disadvantage of this method. Other limitations comprise very short visualization time of haptoglobin-haemoglobin complexes which needs the gel to be immediately recorded and cannot be repeatedly stained.

The development of mass spectrometry in the field of analysis of biopolymers has offered the possibility to use this technique for haptoglobin phenotyping. As the biological material, such as serum, plasma, and other body fluids, is very complex, it is necessary to separate this mixture or enrich the analysed substance before the mass spectrometry analysis. In 1993, William Hutchens developed a new ionization technique called SELDI (Surface-Enhanced Laser Desorption/Ionization) combined with TOF analyser (Time Of Flight-Mass Spectrometry) that uses modified surfaces with separation ability (ion exchange, reverse phase) or selective enrichment with the aid of inorganic stationary phase (IMAC, Immobilized Metal Ion Affinity Chromatography) or with the aid of antibody. In 2004 Stefan Mikkat published the work where the plasma samples were separated first with the use of two- dimensional polyacrylamide electrophoresis and the stained proteins were cleavaged with trypsin and analysed by means of MALDI-TOF-MS (Matrix Assisted Laser Desorption Ionization-Time Of Flight-Mass Spectrometry) (Mikkat, S., Koy, C., Ulbrich, M., Ringel, B. & Glocker, M. O., Proteomics 4, 3921-32 (2004)). This method is very accurate but very time consuming and difficult for routine use in clinical laboratories. In 2004 Jonathan Tolson et al published the work, where they applied the SELDI technique to characterize serum proteins including haptoglobin. For this purpose they used albumin-depleted serum and WCX2 (Weak Cation Exchange) protein chips analysed by means of ProteinChip Reader mass spectrometer (Ciphergen Biosystems, Fremont, Calif. USA). Although some serum proteins were identified by this method, including haptoglobin α1 chain, α2 chain was not successfully identified. The obtained spectra was very complex and due to considerable inaccuracy of the analysis and low sensitivity of the method to haptoglobin detection it is impossible to use this method for haptoglobin phenotyping (Tolson, J. et al., Lab. Invest. 84, 845-56 (2004)).

Kemmons A. Tubbs published another method of determination of haptoglobin phenotype with the use of the combination of affinity chromatography and MALDI mass spectrometry detection. Activated acylimidazole affinity carriers derivatized with anti-haptoglobin antibody, placed in a pipette tip, were used for this purpose. In order to enrich haptoglobin from serum with the use of antibody, the protein was directly eluted from the carrier with the aid of matrix, reduced, and then analysed by MALDI-TOF mass spectrometer in linear mode ((Tubbs, K. a et al. High-throughput MS-based protein phenotyping: application to haptoglobin. Proteomics 5, 5002-7 (2005)). This method is very complex, requiring 17 steps in total and tens washing of affinity tips. Although the pipetting can be automated, the procedure is time consuming, instrumentally and logistically demanding. Furthermore, lengthy purified and accumulated sample of plasma is dosed on the MALDI plate.

Other methods of determination of haptoglobin phenotype comprise the methods based on genotyping. Mikiko Soejima and Yoshiro Koda published the work TaqMan-based real-time PCR for genotyping common polymorphisms of haptoglobin (HP1 and HP2). Clin. Chem. 54, 1908-13 (2008), where they used a real-time method TaqMan PCR for haptoglobin genotyping. This method enables to determine the differences between HP¹ and HP² alleles with the use of the difference between relative number of copies in 1.7-kb area of duplicated HP² by comparing with the intensity of amplified signals of promoter region of a gene.

The patent document WO2008143883 describes the test of haptoglobin genotype based on polymerase chain reaction (PCR) and fluorescent detection with the use of hybridization probe. This essay is costly, as it requires the material for PCR (enzymes, primers, pure chemicals etc.), and time consuming. Further, quite a considerable volume of full blood is required (up to 3 ml). Due to these properties of common PCR in clinical configuration, such as, for example, in the embodiment realized by ARUP Laboratories, the gaining of the information lasts up to three days.

### SUMMARY OF THE INVENTION

The object of the invention is to eliminate the disadvantages of the prior art and to develop the affinity plate, the method of its preparation, and the method of haptoglobin phenotype determination, that would enable the direct application of biological material without its previous preparation or preconcentration. The affinity plate for haptoglobin phenotype determination according to the invention meets these criteria, characterized by comprising the substrate with the surface equipped with haptoglobin antibody in the form of a layer, and dry surface resistivity of less than 10²⁰ Ω·m.

The preferred embodiment of the affinity plate according to the invention is that the substrate surface is conductive with resistivity in the range of 10⁻⁸ to 10¹⁷ Ω·m. The substrate intended for the deposition of haptoglobin antibody is preferably selected from the group comprising conductive metals, their alloys, steel, semi conductible oxides of metals, conductive polymers, conductive forms of carbon, silicon, germanium, glass, indium tin oxide glass, or fused quartz. Thus, the preferable conductivity ranges from well conductive metals and graphene (10⁻⁸ Ω·m) to low conductive fused quartz (10¹⁷ Ω·m).

Another preferable embodiment of the affinity plate according to the invention is the affinity plate equipped directly on the substrate surface with anti-haptoglobin antibody selected from the group comprising polyclonal anti-haptoglobin antibody, monoclonal anti-haptoglobin antibody or single-domain anti-haptoglobin antibody (or nanobody), preferably goat polyclonal anti-haptoglobin antibody or rabbit polyclonal anti-haptoglobin antibody.

The anti-haptoglobin antibody that is deposited directly onto the substrate surface, binds human haptoglobin 1 and human haptoglobin 2.

Important advantage of the affinity plate according to the invention is the bond of the anti-haptoglobin antibody directly onto the conductive substrate surface, without the need of its further fixation or addition of support agents to maintain the peptides activity, as it is known in the prior art, e.g. Jaworek, A., J. Mater. Sci. 42, 266-297 (2006), Lee, B. et al. , Biomaterials 24, 2045-2051 (2003) or Morozov, V. N. & Morozova, T. Y., Deposition of the. 414, 1415-1420 (1999).

The direct binding of anti-haptoglobin antibody is realized by landing the dry ions (multiply charged antibodies without their solvation shell) onto the substrate surface.

This specific binding of antibody onto the substrate surface is enabled by the method of the preparation of the affinity plate according to the invention, where carrier gas and stock solution of the sprayed anti-haptoglobin antibody are introduced, under the pressure of 0.05 to 0.5 Pa, into an enclosed area, where the substrate surface is subjected to the voltage of [±(200-8000) V]. The formed charged aerosol issuing from the electrospray is introduced to evaporation area 10 with the temperature in the range of 30° C. to 80° C. and the issuing dried aerosol is deposited onto the substrate surface 12 placed downstream the evaporation area 10 (see FIG. 1).

The principle of this technique consists in depositing the peptides directly onto the conductive substrate surfaces by means of a special device (see FIG. 1) using electrospray, inert carrier gas, and heated chamber for converting the peptide from the solution to the form of desolvated ions able to react with the substrate surface. After the desolvated ion of antibody lands the substrate surface, the ion charge is discharged and the antibody is immobilized on the substrate surface.

The mask with defined holes of the diameter of 2-3 mm is inserted in front of the substrate surface 12 and thus the discrete circle areas of coherent layer comprising bioactive peptide surface (with the layer of haptoglobin antibody) are formed; the surface is monitored by electron microscopy. Accordingly, it is preferred, when the mask 13, which is under voltage of [±(200-5000) V] of the opposite polarity towards electrospray voltage, is placed between the evaporation area 10 and the substrate surface 12, at the distance up to 3 mm from the substrate surface 12.

An important advantage of the invention consists in the fact that the preparation of the affinity plate proceeds at atmospheric pressure, in short time (in the order of few minutes), and with high efficiency of transferring the peptide onto the surface. Thus, the consumption of anti-haptoglobin antibody during the deposition onto the surface is substantially lower.

As it was mentioned above, the substrate surface 12 of the affinity plate according to the invention is conductive with resistivity of the dry material lower than 10²⁰ Ω·m and is under the voltage of [±(200-5000) V] of the opposite polarity towards the electrospray voltage. Preferably the substrate surface 12 is selected from the group comprising conductive metals, semi conductible oxides of metals, conductive polymers, conductive forms of carbon, silicon, germanium, glass, or steel.

Another preferred embodiment of the preparation of the affinity plate according to the invention uses the carrier gas of the temperature in the range of 30° C. to 80° C., selected from the group comprising nitrous oxide, nitrogen, carbon dioxide, helium, neon, argon, xenon, or oxygen.

The stock solution of the sprayed antibody, preferably used in the method of the affinity plate preparation according to the invention, is aqueous solution or a mixture of water and at least one organic solvent of the content up to 80 vol. %, preferably 1 to 50 vol. %, of e.g. methanol or acetonitrile. It is necessary that the selected organic solvents or their mixtures do not cause precipitation, aggregation, disaggregation, or denaturation of the sprayed anti-haptoglobin antibody.

The preferred concentration of anti-haptoglobin antibody in the stock solution is in the range of 0.01 to 100 μmol/L. Preferably the buffer of the concentration in the range of 1 μmol/L to 1 mol/L can be included.

By the use of syringe piston pressure, the stock solution is pumped into a capillary towards a splitter (common flow rate 0.05 to 5 μL/min). The splitter is closed with micro spray needle (diameter in the range of 1-100 μm). Further, the carrier gas input is connected to the splitter, whereas the pressure of its valve is in the range of 0.05 to 0.5 MPa. The carrier gas can be introduced at the room temperature or preheated up to 80° C. Preferably it is preheated at the temperature in the range of 30 to 40° C. A high voltage source [±(500-8000)V] is a part of the electrospray section and it is connected to the electrospray section in any conductive area. The evaporation area can be externally heated up to 80° C., depending on thermostability of anti-haptoglobin antibody to prevent its denaturation or loss of its activity. The end part comprises the surface for the modification in front of which a mask can be placed; the mask is grounded or connected to the second high voltage source (from 0 to ±8000V). In case the mask is not applied, the voltage can be connected directly to the surface for the modification. The distance of the mask from the evaporation area outlet is less than 20 mm and more than 1 mm from the surface for the modification In case the mask is not applied, the distance of the surface from the evaporation area outlet is less than 50 mm.

The process of deposition of the antibody proceeds as follows: the high voltage source is connected to the electrospray part. The solution of anti-haptoglobin antibody (A) of the flow rate in the range of 0.01 to 50 μL/min is introduced into the stream of the pressured (0.05 to 0.5 MPa) carrier gas, which is inert gas, preferably nitrogen, argon, helium, or neon. Due to the high voltage (+/− 200-4000V) and the stream of pressurized carrier gas, the solution of a peptide (A) is electronebulized from the spray needle to form a charged aerosol (B). The charged aerosol is then introduced into the evaporation area where the aerosol is dried.

The aerosol, excited in this manner, transforms into a dry aerosol and then into an ion beam (C). In addition, the dried charged particles—ions (C) can be focused by means of high voltage (+/− 200-4000V), of the polarity opposite towards to the electrospray voltage, brought to the mask or directly onto the surface, and thanks to the energy gained by rapid heating they are bound to the modified surface where they form macroscopically homogenous print, whose shape and size is defined by the shape and the size of the holes in the mask. If the mask is not applied, the print size and shape is defined by the section shape of the evaporation area.

The affinity plate according to the invention, prepared in this manner, can be used as a part of the kit (i.e. diagnostic kit) for the determination of haptoglobin phenotype by means of desorption ionization techniques selected from the group comprising MALDI (matrix-assisted laser desorption/ionization), SALDI (surface-assisted laser desorption/ionization), NALDI (nanostructure-assisted laser desorption/ionization), GALDI (graphite-assisted laser desorption/ionization) or similar LDI (laser desorption/ionization) technique. Further, the desorption ionization technique, where the analysed surface is placed outside the mass spectrometer, for example DART (Direct Analysis in Real Time) or DESI (desorption electrospray ionization), can be used.

The affinity plate according to the invention enables haptoglobin to be specifically preconcentrated from biological material (the sample taken from the subject, for example human or animal), which represent a complex mixture. Binding of haptoglobin to the antibody presents specific affinity reaction between them to form their complex that remains on the surface of the affinity plate after washing the sample matrix.

Another embodiment of the invention is the method of the determination of haptoglobin phenotype characterized in that the biological material is deposited on the layer formed by anti-haptoglobin antibody bound on the affinity plate according to the invention. After washing the plate, aqueous solution of reducing agent is added and the presence of a and 13 subunits of haptoglobin is determined by means of mass spectrometry desorption ionization techniques.

According to the preferred embodiment of the determination method according to the invention the biological material is selected from the group comprising plasma, serum, hemolysate, or blood, their chemically processed forms or their buffer solutions (concentration range of 1 μM to 1 M), the biological material being applied onto the affinity plate in the amount of 0.5 to 10 μL.

According to another preferred embodiment of the determination method according to the invention, after the application of the biological material onto the layer of anti-haptoglobin antibody, the affinity plate with biological material is incubated for 5 minutes to 24 hours at the temperature of 10 to 50° C. and then it is washed with buffer.

According to another the preferred embodiment of the determination method according to the invention, after adding the aqueous solution of reducing agent, the affinity plate is incubated for 5 to 30 minutes at the temperature of 20-37° C., the reducing agent being selected from the group comprising tris(2-carboxyethyl)phosphine, mercaptoethanol, or dithiothreitol. Concentration of the reducing agent in aqueous solution is preferably in the range of 5 to 100 mmol/L.

According to another preferred embodiment of the determination method according to the invention, after adding the aqueous solution of reducing agent, the affinity plate is left to incubate for 5 to 30 minutes at the temperature of 20 to 37° C., and then the solution of MALDI matrix is added and left to dry at the temperature of 10 to 50° C. 0.5 to 10 μL of serum, plasma, hemolysate, blood, or their diluted form in suitable buffer is applied onto the anti-haptoglobin antibody layer placed on the affinity plate according to the invention and is incubated for 5 min to 24 hours at the temperature of 10° C. to 50° C.

To prevent too fast evaporation of the analyte, the affinity plate is placed into an enclosed area, preferably is covered by Petri dish. After the incubation, the surface with bound haptoglobin is washed with buffer for 3×10 min, preferably PBS or another buffer can be used. As the presence of a subunits that are associated with β subunit via disulphide bridges is monitored by means of mass spectrometry, it is necessary to reduce the bound haptoglobin with the use of reducing agent to free the analysed a subunits. Preferably aqueous solution of TCEP (tris(2-carboxyethyl)phosphin), mercaptoethanol, or DTT (dithiothreitol) of the concentration 5 to 100 mmol/L can be used for the reduction by applying it directly onto the surface with biological material (analyte). If the MALDI method is used for the determination of haptoglobin phenotype, the solution of ionization matrix used for enhancing the ionization efficiency of the analyte, described hereinafter, is added after 5 to 30 minutes of incubation at the room temperature. Ionization MALDI matrix, mediating ionization of haptoglobin molecules, further called the matrix, is a compound that absorbs laser radiation and enables desorption and charging of the analyte during excitation, protecting the analyte from the direct contact with laser radiation. Preferably low molecular organic derivatives of aromatic acids (in the range of 150-400 Da), for example of benzoic acid or cinnamic acid, are used as the matrixes. These matrixes are preferably 2,5-dihydroxybenzoic acid (DHB), alpha-cyano-4-hydroxycinnamic acid (CHCA), sinapic acid (SA), 2,5-dihydroxyphenylmethylketone (DHAP), and ferulic acid (FA). There are various solvents of the matrix. Most often, the solvent comprises aqueous mixture of organic solvent (ethanol, methanol, or acetonitrile) and acid, for example trifluoroacetic acid. Water content is in the range of 20-80% (v/v), completed with organic solvent. The acid is added in the range of 0.1-1% (v/v). Nowadays, the matrix DHAP is preferably used for protein determination by the MALDI method. The matrix is applied in aforementioned solvent system and is left to dry at the temperature of 10° C. to 50° C. The formed matrix crystals with the analyte on the surface enable its ionization and following analysis, for example by the means of MALDI MS.

The application of the ionization matrix matters with the use of the MALDI method only. The other desorption ionization techniques suitable for protein ionization usually do not gain substantially improved ionization by the use of an organic matrix. The methods without matrix were described in the literature, for example Peterson D.S.:, Mass Spectrometry Reviews, 2007, 26, 19-34.

The object of the invention is also the kit (further “diagnostic kit”) comprising the affinity plate according to the invention together with agents necessary for performing the method of the determination of haptoglobin phenotype according to the invention.

In the mass spectra, there can be observed the signals corresponding to al chain only (approximately mlz 9190) in the case of phenotype 1-1, or to α2 chain only (approximately m/z 15940) for phenotype 2-2. Both the signals corresponding to α1 and α2 chain are observed for phenotype 2-1 (FIG. 2). In the case of using the blood or hemolysate, also other signals corresponding to haemoglobin a and haemoglobin β molecules, of the molecular weight of 15122 or 15863 respectively (FIG. 3), are present in the spectra.

The modification of the surface with the protein was proved also by means of scanning electron microscopy (SEM). FIG. 4 shows the surface coated with the antibody immediately after performing the deposition and then after its washing with buffer. The scanning electron microphotography apparently shows that even after washing, a thin layer of peptide molecules remains captured on the surface, which is consistent with affinity activity of the surface modified in this manner.

The Western blot method was used to verify the accuracy of phenotype determination by means of affinity surfaces combined with mass spectrometry detection. One microliter of the serum was mixed with 10 μL of reducing sample buffer and then applied on gradient (4-15%) polyacrylamide gel at the presence of sodium dodecyl sulphate (SDS). The separated proteins were transferred onto a nitrocellulose membrane by means of electron transfer. The membrane was incubated with polyclonal anti-haptoglobin antibody and then with secondary antibody conjugated with horseradish peroxidase. The proteins were visualized by means of chemiluminiscence (FIG. 5).

### Terms Definitions

The term “substrate surface” means any solid surface having at least minimum conductivity. The criterion of minimal conductivity of the substrate surface, for the purposes of this invention, is the resistivity lower than 10¹⁷ Ω·m, for dry substrate surface at the temperature of 20° C. These surfaces comprise mainly conductive metals and semi conductible oxides of metals, steel, but also for example glass, fused quartz, indium tin oxide glass, silicon, germanium, conductive polymers, and conductive forms of carbon.

The term “haptoglobin” refers to human haptoglobin 1 or human haptoglobin 2.

The term “anti-haptoglobin antibody” refers to the immune system peptide commonly called immunoglobulin that is able to bind haptoglobin molecules, i.e. the molecules of human haptoglobin 1 or human haptoglobin 2. Thus, the anti-haptoglobin antibody refers to any of immunoglobulins selected from the group comprising immunoglobulins of main groups (IgA, IgD, IgE, IgG, IgM) or their sub groups (for example IgG1, IgA2 and the like), as well as their functional fragments, such as Fab or Fv. Further, the antibody refers also to single domain antibody (also called nanobody) or fusion peptide able to selectively bind haptoglobin (for example single chain antibodies scFv and Fc fusion peptides). The antibody can be polyclonal antibody derived from mammals, mainly humans, mice, rabbits, goats, donkeys, horses, camels, or llamas, or monoclonal derived from mammals, mainly humans, mice, rabbits, or their respective cell lines.

Examples of polyclonal anti-haptoglobin antibodies from various organisms are listed in

Preferred embodiments of the invention. IgG1 mice monoclonal antibody, clone HG-36, is a suitable example of monoclonal anti-haptoglobin antibody. This monoclonal antibody can be purchased for example from Abeam, Sigma-Aldrich or GeneTex.

The term “layer” refers to a continuous layer of antibody deposited on the substrate surface. The substrate surface can be covered partially by this layer, for example due to the deposition of the antibody through a mask.

The term “buffer” refers to conjugated pair of acid (or base) of the concentration from 1 μmol/L to 1 mol/L, where the anion is preferably selected from the group comprising CO₃^{2-,} CH₃COO⁻, , HCOO⁻, Cl⁻, and the cation is preferably selected from the group comprising H⁺, Na⁺, K⁺, NH₄³⁰, triethanolamine, trimethylamine, triethylamine or pyridine; preferably PBS (Phosphate Buffered Saline), HEPES (4-(2-hydroxyethyl)-1-piperazinethansulfonic acid can be used.

The term “conductive polymers” generally refers to polymer compounds meeting the above mentioned condition of conductivity of the substrate surface. They are mostly thermoplastics, for example polyacetylene, polythiophene, or polyphenylvinylene, and the materials derived from them.

The term “conductive forms of carbon” refers to carbon materials capable to conduct electric current, for example graphite, graphene, or carbon nanotubes, meeting the abovementioned condition of conductivity of the substrate surface.

The term “room temperature” means 25° C.

### BRIEF DESCRIPTION OF THE DRAWINGS

FIG. 1: The apparatus for the preparation of the affinity plate for the determination of haptoglobin phenotype with the use of desorption ionization mass spectrometry comprises T-splitter 1, syringe pump 2 operating the piston 3 of the syringe 4 with stock solution A, tube 5, microspray needle 6 on the splitter 1, carrier gas inlet 7 with the possibility of preheating, high voltage source 8 for electrospray connected to the conductive part 9 of the syringe 4, evaporation area 10, surface 12 for modification, high voltage source 11 connected to substrate surface 12. When the stock solution passes the spray, charged aerosol B is formed and transformed into dry aerosol/ion beam C by passing the tube which can be optionally heated.

FIG. 2: MALDI mass spectra of haptoglobin a chains after serum incubation with polyclonal anti-human haptoglobin antibody _{[MD1]}that was applied onto the surface. Haptoglobin is enriched after washing and its a chains can be identified in spectra after the preceding reduction of disulphide bridges. The figure shows mass spectra of three possible combinations of phenotypes: Hp1-1; Hp2-1; Hp2-2 (in the order down from top).

FIG. 3: MALDI mass spectra of haptoglobin a chains after incubation of hemolysate with polyclonal anti-human haptoglobin antibody, applied onto the surface. Haptoglobin is enriched after washing and its a chains can be identified in spectra after the preceding reduction of disulphide bridges. The figure shows mass spectra of three possible combinations of phenotypes: Hp1-1; Hp2-1; Hp2-2 (down from top respectively).

The signals corresponding to the molecules of α and β haemoglobin can be observed in spectra due to the formation of haptoglobin-haemoglobin complex. Haemoglobin is not present in serum spectra in the FIG. 2, because the blood cells—erythrocytes containing haemoglobin are removed during the preparation of serum.

FIG. 4: The pictures of surfaces modified with anti-haptoglobin antibody obtained by means of electron microscopy. Right—the surface before washing; formed with the layer of antibody and the layer of salt. Left—the surface after washing with distilled water. Black coloured spots present the applied antibody.

FIGS. 5a to 5d: The results of analysis of determination of haptoglobin phenotype for particular samples by means of immunostaining with anti-haptoglobin antibody after the separation by means of gel electrophoresis (the photograph inserted at the top part) and by means of enrichment on the affinity surface with mass spectrometry detection. It can be seen from the picture 5a that both the methods give the same results. Mass spectra shows the presence of α1 and α2 chains of haptoglobin in the form of spectral peaks, while gel electrophoresis in the form of electrophoretic zones (bands). It can be seen from the picture that the graphical presentation by means of spectrometric peaks and electrophoretic bands is the same for each of the samples 1-9, which indicates equivalency of both the techniques. The results of the analysis of determination of haptoglobin phenotype for the samples 10-36 are presented in FIGS. 5b to 5d.

FIG. 6: A) Mass spectra of haptoglobin a chains after the incubation of the serum with polyclonal anti-human haptoglobin antibody, provided by Fitzgerald Company, that was applied onto Indium Tin Oxide surface. The figure shows mass spectra of phenotype Hp2-1. B) Mass spectra of haptoglobin a chains after the incubation of the serum with polyclonal anti-human haptoglobin antibody, provided by Sigma-Aldrich Company, that was applied onto fused quartz. The figure shows mass spectra of phenotype Hp2-1. C) Mass spectra of haptoglobin a chains after the incubation of the serum with polyclonal anti-human haptoglobin antibody, provided by Fitzgerald Company, that was applied onto stainless surface. The figure shows mass spectra of phenotype Hp2-1.

### PREFERRED EMBODIMENTS OF THE INVENTION

### Example 1—The Preparation of the Affinity Plate for the Determination of Haptoglobin Phenotype (Surface: Stainless Steel; Resistivity: 10⁻⁸ Ω·m; Antibody Used: Anti-Haptoglobin, Fitzgerald Company; Organism: Goat)

The modified surface was prepared by electrospray deposition with dry ions landing onto the surface for 20 minutes according to the following procedure:

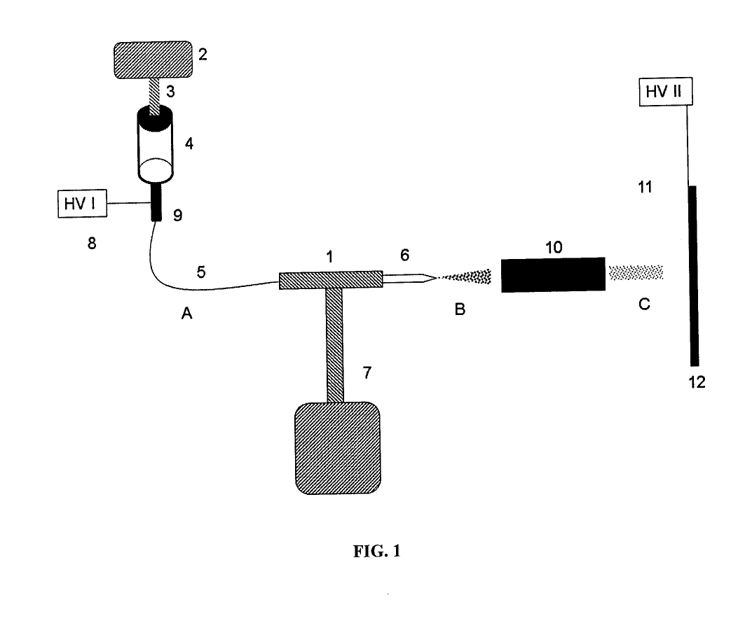

The values on the apparatus according to FIG. 1 were set as follows:

Flow rate of the pump 2: 1 μL/min
Voltage of the source 8 brought on the conductive part 9 of the syringe 4: 1500V
Temperature of the evaporation area of the tube shape 10: 40° C.
Voltage from the source 11 on the mask 13: −1500V
Pressure on the carrier gas inlet 7: 0.25 MPa
Carrier gas: nitrogen
Temperature of the carrier gas: 40° C.
Shape of the hole on the mask: circle of the diameter of 2 mm

Syringe pump 2 was filled with the solution of anti-haptoglobin antibody of the concentration of 2 μmol/L in 5 mmol/L ammonium acetate, 30 vol. % acetonitrile (Solution A). High voltage source 8 was connected to the conductive part 9 of the syringe 4 with stock solution that was connected via capillary 5 to the splitter 1. The solution of antibody (A) was introduced into the splitter by the syringe pump 2, where it was electronebulized from the spray needle due to the high voltage and the flow of pressurized carrier gas from the inlet to form the charged aerosol (B). The formed aerosol was introduced into the area 10 of the tube shape of the diameter 5 mm where the aerosol was dried and further passed through mask 13 towards the surface from stainless steel 12. After finishing the process, high voltage from both the sources 8 and 11 was turned off, the surface was removed and washed with water. 240 pmol of antibody was used for this method and the formed layer was of a circle shape of the diameter given by the mask (2 mm).

According to the abovementioned method, the surfaces were modified with anti-haptoglobin antibody. The particular values set on apparatuses are stated individually in each example. The processes differed in other parameters:

1) Antibody concentration: A) 0.01 μmol/L, B) 1 μmol/L, and C) 100 μmol/L.
2) Antibodies were obtained from various companies, mainly Fitzgerald and Sigma-Aldrich
3) The temperature of the evaporation area during deposition of antibody was in the range of 25-45° C.
4) The surface of the modified plate was from A) stainless steel, B) ITO glass (Indium Tin Oxide), C) aluminium.

### Example 2 (Anti-Haptoglobin Antibody: Fitzgerald; Immunised Organism: Goat; Starting Material: Hemolysate, Surface: Indium Tin Oxide; resistivity: 10⁻⁴ Ω·m)

The modified surface was prepared by preforming the electrospray deposition according to Example 1, by landing dry ions of polyclonal anti-haptoglobin antibody of the concentration of 2 μmol/L on ITO (Indium Tin Oxide) glass for 5 minutes and at the temperature of the evaporation area 40° C.

The values on the apparatus according to FIG. 1 were set as follows:

Flow rate of the pump: 0.54/min
Voltage of the source 8 brought on the conductive part 9 of the syringe 4: 1300V
Temperature of the evaporation area of the tube shape 10: 40° C.
Voltage from the source 11 on the mask 13: −1000V
Pressure on the carrier gas inlet 7: 0.25 MPa
Carrier gas: nitrogen
Temperature of the carrier gas: 30° C.
Shape of the hole on the mask: circle of the diameter of 2 mm

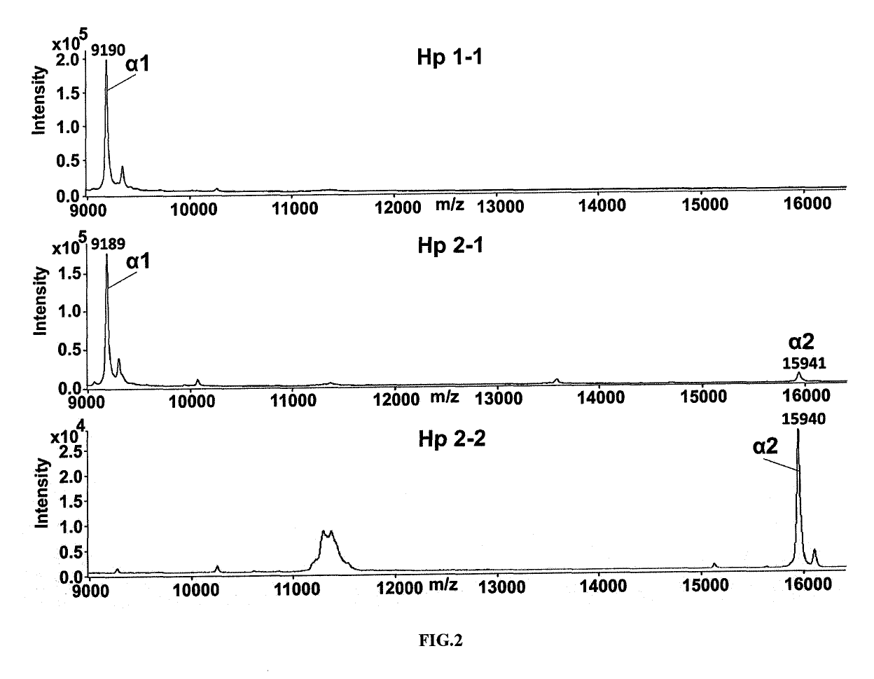

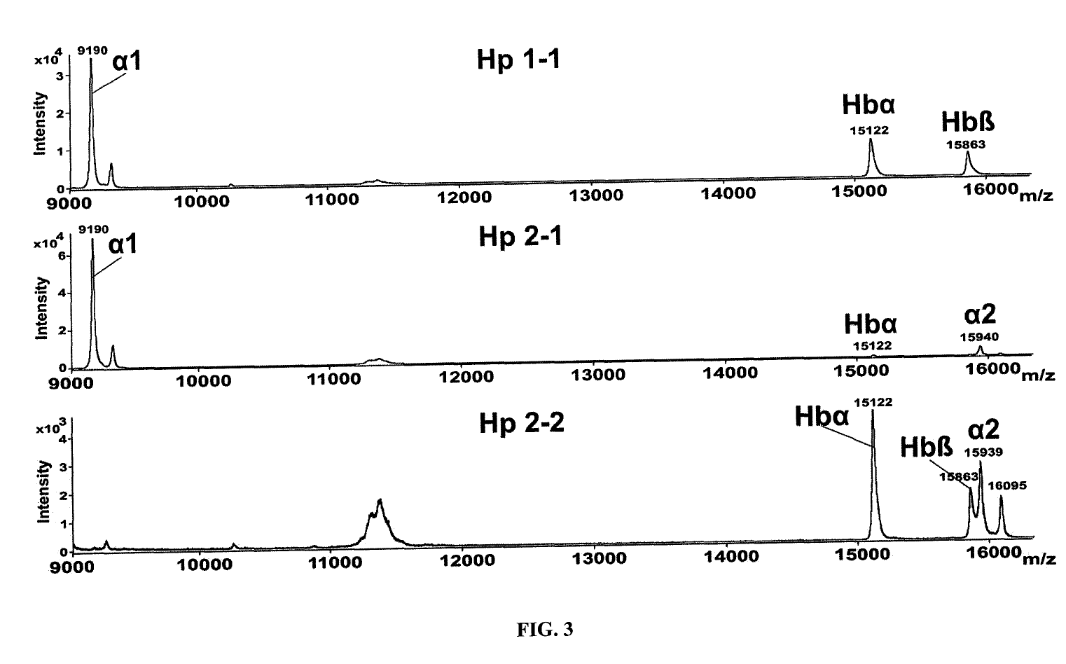

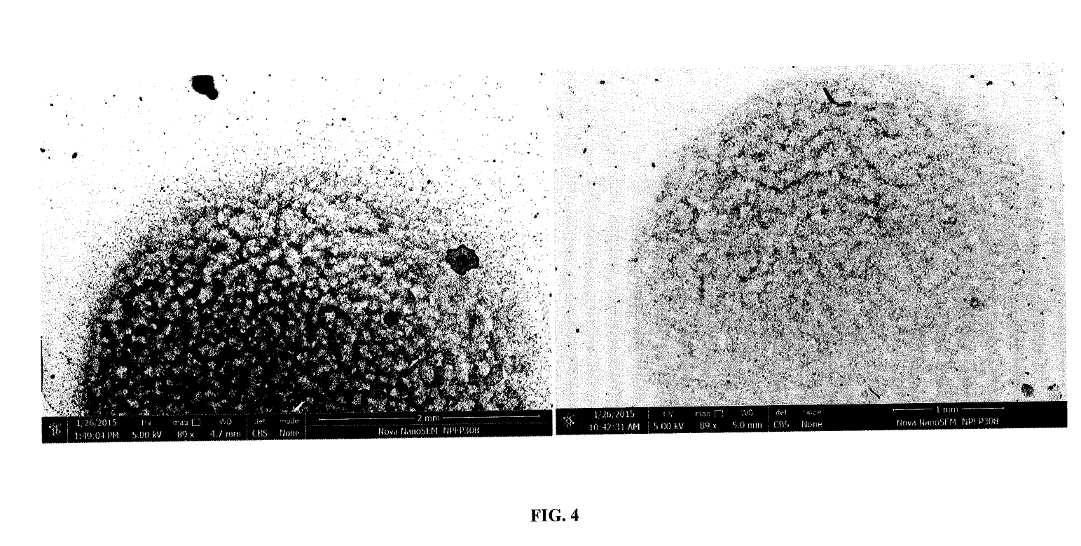

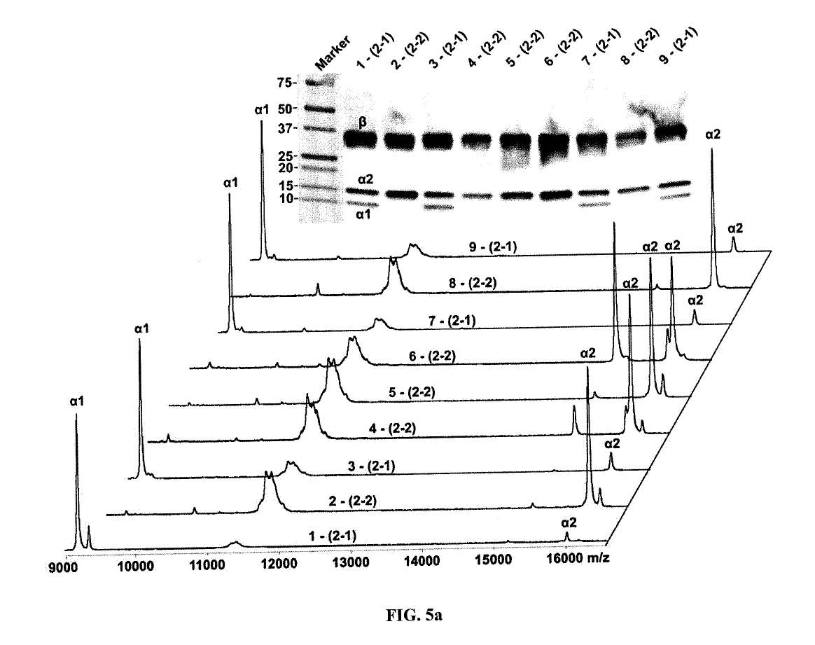

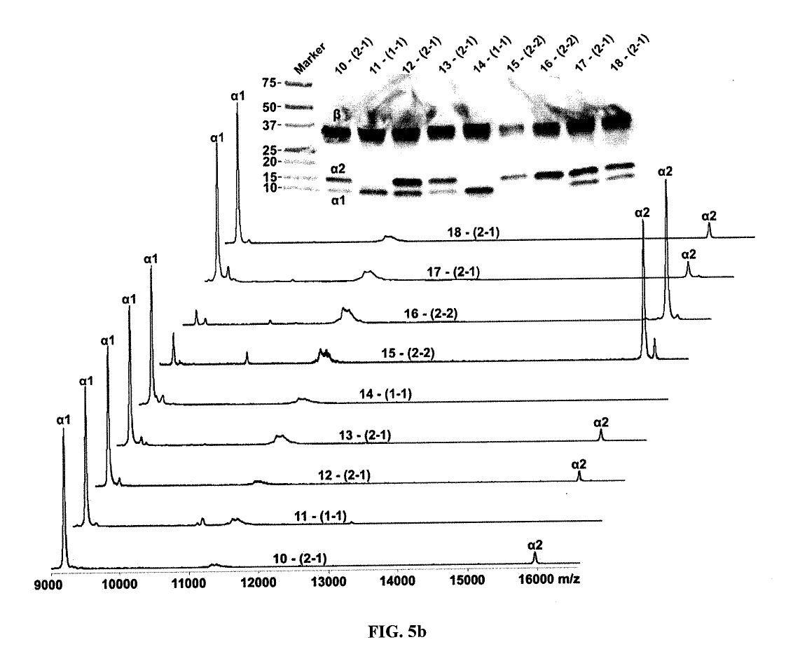

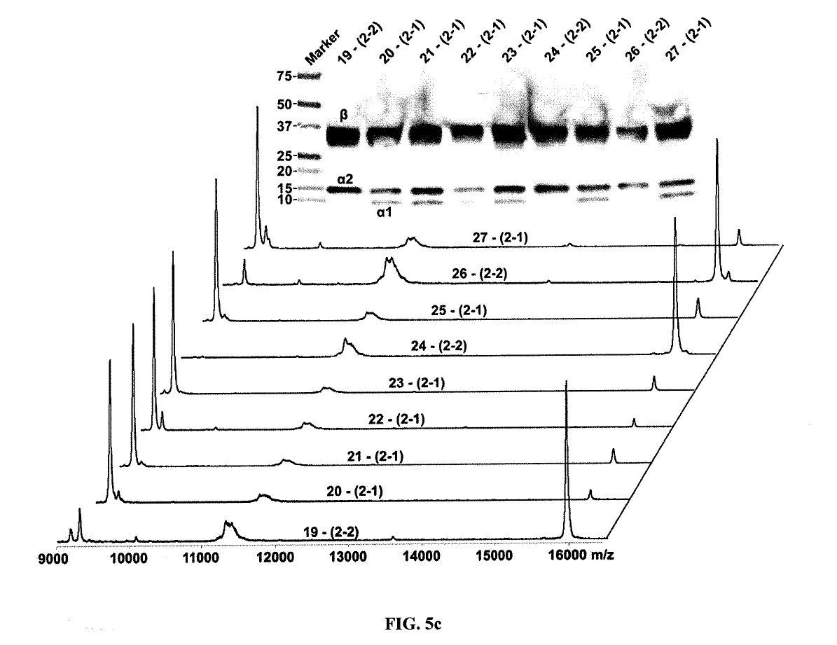

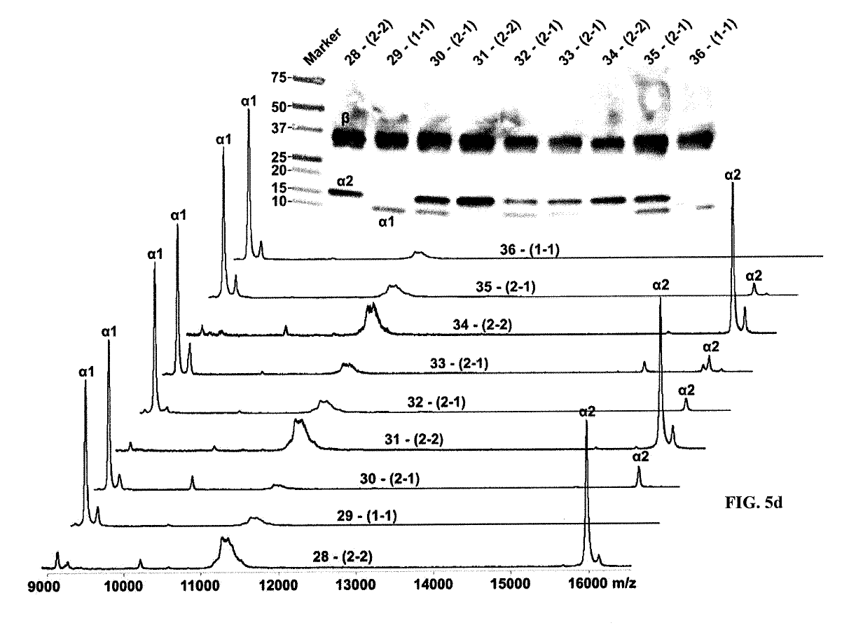

After washing the glass with water, 2 μL of hemolysate were deposited on the sites with bound antibody and left to incubate in Petri dishes for 1 h at the room temperature. Then the surface was washed for 3×10 min with 1× PBS solution, pH 7.2, and then with distilled water for 1×5 min. After the surface dried, 1 μL of 15 mmol/l aqueous solution of TCEP was added and the surface was incubated for 30 min in Petri dish at the room temperature. After removing the surface from the Petri dish, matrix was added to the samples with bound haptoglobin, the matrix having following composition: 7.6 mg of DHAP dissolved in 375 μL, of ethanol +125 μL of DHAC (diammonium hydrogen citrate) of the concentration of 18 mg/mL. This matrix solution was mixed with 0.1% trifluoroacetic acid in the ratio of 1:1. One microliter of the matrix was mixed with the sample directly on the surface and left to crystalize at the temperature of 35° C. The samples were analysed by means of MALDI mass spectrometry (FIG. 2).

### Example 3 (Anti-Haptoglobin Antibody: Fitzgerald Company; Immunised Organism: Goat; Starting Material: Serum, Surface: ITO; Resistivity: 10⁻⁴ Ω·m)

The modified surface was prepared by preforming the electrospray deposition according to Example 1, by landing the dry ions of polyclonal anti-haptoglobin antibody (source: Fitzgerald Company, organism: goat) of the concentration of 2 μmol/L, on the ITO (Indium Tin Oxide) glass for 5 minutes, whereas the temperature of the evaporation area during substance deposition was 40° C.

The values on the apparatus according to FIG. 1 were set as follows:
Flow rate of the pump: 2 μL/min
Voltage of the source 8 brought on the conductive part 9 of the syringe 4: 1700V
Temperature of the evaporation area of the tube shape 10: 35° C.
Voltage from the source 11 on the mask 13: −1200V
Pressure on the carrier gas inlet 7: 0.25 MPa
Carrier gas: nitrogen
Temperature of the carrier gas: 40° C.
Shape of the hole on the mask: circle of the diameter of 2 mm

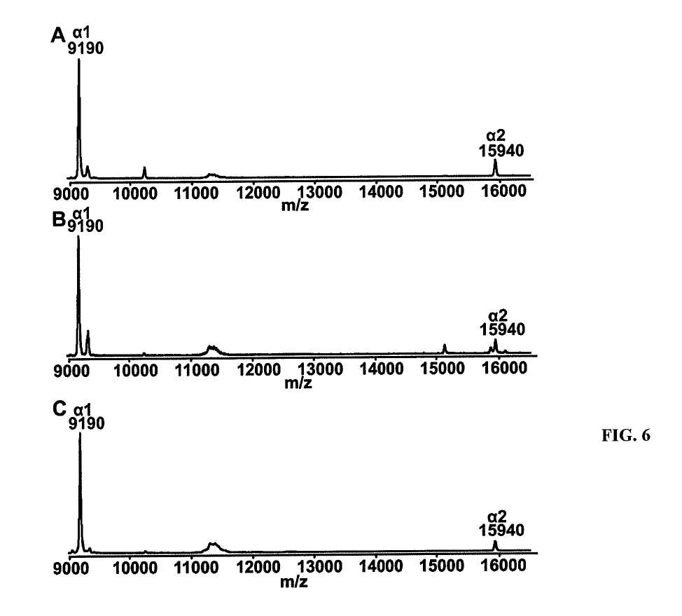

After washing the glass with water, 2 μL of serum were deposited on the sites with bound antibody and left to incubate in Petri dishes for 1 h at the room temperature. Then the surface was washed for 3×10 min with 1× PBS solution, pH 7.2, and then with distilled water for 1×5 min. After the surface dried, 1 μL of 50 mM aqueous solution TCEP was added and the surface was incubated for 30 min in Petri dish at the room temperature. After removing the surface from the Petri dish, matrix was added to the samples with bound haptoglobin, the matrix having following composition: 7.6 mg of DHAP dissolved in 375 μL of ethanol +125 μL of DHAC (diammonium hydrogen citrate) of the concentration of 18 mg/mL. This matrix solution was mixed with 0.1% trifluoroacetic acid in the ratio of 1:1. One microliter of the matrix was mixed with the sample directly on the surface and left to crystalize at the room temperature. The samples were analysed by means of MALDI mass spectrometry (FIG. 6A).

### Example 4 (Anti-Haptoglobin Antibody: Sigma-Aldrich; Immunised Organism: Rabbit; Starting Material: Serum, Surface: Fused Quartz; Resistivity: 10¹⁷ Ω·m)

The modified surface was prepared by preforming the electrospray deposition according to Example 1, by landing the dry ions of polyclonal anti-haptoglobin antibody (Sigma-Aldrich) of the concentration of 3 μmol/L on the fused quartz for 5 minutes and at the temperature of the evaporation area of 40° C.

The values on the apparatus according to FIG. 1 were set as follows:

Flow rate of the pump: 1 μL/min
Voltage of the source 8 brought on the conductive part 9 of the syringe 4: 1400V
Temperature of the evaporation area of the tube shape 10: 35° C.
Voltage from the source 11 on the mask 13: −1400V
Pressure on the carrier gas inlet 7: 0.25 MPa
Carrier gas: nitrogen
Temperature of the carrier gas: 35° C.
Shape of the hole on the mask: circle of the diameter of 2 mm

After washing the glass with water, 2μL of serum were deposited on the sites with bound antibody and left to incubate in Petri dishes for 1 h at the room temperature. Then the surface was washed for 3×10 min with 1× PBS solution, pH 7.2, and then with distilled water for 1×5 min. After the surface dried, 1 μL of 50 mmol/L TCEP aqueous solution was added and the surface was incubated for 30 min in Petri dish at the room temperature. After removing the surface from the Petri dish, matrix was added to the samples with bound haptoglobin, the matrix having following composition: 7.6 mg of DHAP dissolved in 375 μL of ethanol +125 μL of DHAC (diammonium hydrogen citrate) of the concentration of 18 mg/mL. This matrix solution was mixed with 0.1% trifluoroacetic acid in the ratio of 1:1. One microliter of the matrix was mixed with the sample directly on the surface and left to crystalize at the room temperature. The samples were analysed by means of MALDI mass spectrometry (FIG. 6B).

### Example 5 (Anti-Haptoglobin Antibody: Fitzgerald; Immunised Organism: Goat; Starting Material: Serum, Surface: Stainless; Resistivity: 10⁻⁸ Ω·m)

The modified surface was prepared by preforming the electrospray deposition according to Example 1, by landing the dry ions of polyclonal anti-haptoglobin antibody (Fitzgerald) of the concentration of 4 μmol/L on the stainless surface for 5 minutes and at the temperature of the evaporation area of 40° C.

The values on the apparatus according to FIG. 1 were set as follows:

Flow rate of the pump: 2.5 μL/min
Voltage of the source 8 brought on the conductive part 9 of the syringe 4: 1500V
Temperature of the evaporation area of the tube shape 10: 60° C.
Voltage from the source 11 on the mask 13: −1600V
Pressure on the carrier gas inlet 7: 0.25 MPa
Carrier gas: argon
Temperature of the carrier gas: 35° C.
Shape of the hole on the mask: circle of the diameter of 2 mm

After washing the stainless with water, 2 μL of serum were deposited on the sites with bound antibody and left to incubate in Petri dishes for 1 h at the room temperature. Then the surface was washed for 3×10 min with 1× PBS solution, pH 7.2, and then with distilled water for 1×5 min. After the surface dried, 1 μL of 50 mmol/L TCEP aqueous solution was added and the surface was incubated for 30 min in Petri dish at the room temperature. After removing the surface from the Petri dish, matrix was added to the samples with bound haptoglobin, the matrix having following composition: 7.6 mg of DHAP dissolved in 375 μL of ethanol +125 μL of DHAC (diammonium hydrogen citrate) of the concentration of 18 mg/mL. This matrix solution was mixed with 0.1% trifluoroacetic acid in the ratio of 1:1. One microliter of the matrix was mixed with the sample directly on the surface and left to crystalize at the room temperature. The samples were analysed by means of MALDI mass spectrometry (FIG. 6C).

### Example 6 (Anti-Haptoglobin Antibody: fitzgerald; Immunised Organism: Goat; Starting Material: Serum, Surface: Porous Silicon; Resistivity: 10² Ω·m; without the Presence of Matrix and without the Incubation)

The modified surface was prepared by preforming the electrospray deposition according to Example 1, by landing the dry ions of polyclonal anti-haptoglobin antibody (Fitzgerald) of the concentration of 2.5 μmol/L on the porous silicon surface for 5 minutes and at the temperature of the evaporation area of 40° C.

The values on the apparatus according to FIG. 1 were set as follows:

Flow rate of the pump 1 μL/min
Voltage of the source 8 brought on the conductive part 9 of the syringe 4: 1500V
Temperature of the evaporation area of the tube shape 10: 37° C.
Voltage from the source 11 on the mask 13: −300V
Pressure on the carrier gas inlet 7: 0.5 MPa
Carrier gas: helium
Temperature of the carrier gas: 20° C.
Shape of the hole on the mask: circle of the diameter of 2 mm

After washing the surface with water, 1 μL of serum was deposited on the sites with bound antibody. Then the surface was washed for 3×10 min with 1× PBS solution, pH 7.2, and then with distilled water for 1×5 min. After the surface dried, 1 μL of 50 mmol/L TCEP aqueous solution was added to the samples. The surface was washed with 1% aqueous solution of formic acid. The samples were analysed by means of MALDI mass spectrometry without the presence of the ionization matrix.

1. The affinity plate for the determination of haptoglobin phenotype characterized in that it consists of the substrate, the surface of the substrate being provided with anti-haptoglobin antibody in the form of a layer and its resistivity is lower than 10²⁰ Ω·m.

2. The affinity plate according to the claim 1 characterized in that the resistivity of the substrate surface is in the range of 10⁻⁸ to 10¹⁷ Ω·m, the substrate being selected from the group comprising conductive metals, alloys thereof, steel, semi conductible oxides of metals, conductive polymers, conductive forms of carbon, silicon, germanium, glass.

3. The affinity plate according to the claim 1 or claim 2 characterized in that the anti-haptoglobin antibody is selected from the group comprising polyclonal anti-haptoglobin antibody, monoclonal anti-haptoglobin antibody or single-domain anti-haptoglobin antibody, preferably goat polyclonal anti-haptoglobin antibody or rabbit polyclonal anti-haptoglobin antibody.

4. The kit for the determination of haptoglobin phenotype characterized in that it comprises the affinity plate according to any of the claim 1 to 2.

5. The method of the determination of haptoglobin phenotype characterized in that it comprises the steps of:
a) depositing a biological material on the layer consisting of the anti-haptoglobin antibody, lying on the affinity plate according to any of the claims 1 to 2, then it is washed at least once with buffer,
b) adding an aqueous solution of the reducing agent, and
c) detection of the presence of a and β subunits of haptoglobin by means of desorption ionization mass spectrometry techniques.

6. The method according to the claim 5 characterized in that the biological material is selected from the group comprising plasma, serum, hemolysate, or blood, or solutions thereof in buffer.

7. The method according to the claim 5 or claim 6 characterized in that the amount of the deposited biological material is in the range of 0.5 to 10 μl.

8. The method according to the claim 5 characterized in that after the step a) the affinity plate with biological material is incubated for 5 minutes to 24 hours at the temperature of 10 to 50° C. and then it is washed with buffer.

9. The method according to the claim 5 characterized in that after the step b) the affinity plate with reducing agent is incubated for 5 minutes to 30 minutes at the temperature 20° C. to 37° C., the reducing agent being selected from the group comprising tris(2-carboxyethyl)phosphin, mercaptoethanol, or dithiothreitol.

10. The method according to the claim 5 characterized in that the concentration of the reducing agent in aqueous solution is in the range of 5 to 100 mmol/L.

11. The method according to the claim 9 or claim 10 characterized in that after the incubation of the affinity plate with the reducing agent the solution of ionization MALDI matrix is added, then it is left to dry at the temperature of 10° C. to 50° C.

12. The method according to the claim 11 characterized in that the ionization MALDI matrix is selected from the group comprising 2,5-dihydroxybenzoic acid, alpha-cyano-4-hydroxycinnamic acid, sinapic acid, 2,5-dihydroxyphenylmethylketone, and ferulic acid.
